(19) United States
(12) Patent Application Publication
(10) Pub. No.: US 2020/0277665 A1
(43) Pub. Date: Sep. 3, 2020

(54) REVERSIBLE THERMODYNAMIC TRAP (THERMOTRAP) IN AMPLIFICATION OF NUCLEIC ACIDS

(71) Applicant: DNAe Diagnostics Ltd, London (GB)

(72) Inventors: Kamil Andrzej LIPINSKI, London (GB); Francois Joel PAILLIER, London (GB); Brian James MCKEOWN, London (GB)

(73) Assignee: DNAe Diagnostics Ltd, London (GB)

(21) Appl. No.: 16/650,209

(22) PCT Filed: Sep. 24, 2018
(86) PCT No.: PCT/GB2018/052708

§ 371 Date: Mar. 24, 2020

(30) Foreign Application Priority Data
Sep. 25, 2017 (GB) 1715465.9

(51) Int. Cl. C12Q 1/6848; C12Q 1/6853

(52) U.S. Cl. CPC C12Q 1/6848; C12Q 1/6853; C12Q 2525/197; C12Q 2525/179; C12Q 2525/186; C12Q 2525/161

(57) ABSTRACT

Described is a method and kit for efficiently amplifying and detecting certain nucleic acid sequences from a population. The invention is intended to provide increased assay specificity by minimizing unwanted interactions between priming oligonucleotides (primers).

### FIELD OF THE INVENTION

The present invention relates to the design of molecular biology assays based on nucleic acid amplification, such as, but not limited to, Polymerase Chain Reaction (PCR) and various isothermal amplification methods. The invention is intended to provide increased assay specificity by minimizing unwanted interactions between priming oligonucleotides (primers).

### BACKGROUND TO THE INVENTION

Achieving high specificity, sensitivity and product yield is crucial in highly multiplexed molecular biology assay where many nucleic acid sequences are amplified at once in a single reaction. Tens, hundreds, or thousands of oligonucleotide primers can be mixed to perform amplification of one or several clinically-relevant targets in one assay. As the level of multiplexing increases, so does the combinatorial complexity of primer interactions, and some thermodynamic bottlenecks appear that lead to decreased sensitivity or even false-positive results.

A typical example is the unwanted amplification of primer dimers due to non-specific cross-reactivity of primers. Numerous biochemical, biophysical, and bioinformatic methods have been developed to aid in assay design by minimizing the probability of unwanted primer interactions. Many focus on preventing amplification from initiating before all reagents are present and the reaction is brought to its desired temperature (e.g. ‘hot start’ amplification). In terms of computational approaches, pools of candidate primers are screened for complementarity at their 3′-ends and thermodynamic stability of primer duplexes in silico is calculated. Even though primers can be designed such that there is virtually no complementarity between them that could facilitate dimer formation through base-pairing, primer dimers may still form in a sequence-independent manner. There is very limited evidence that would shed light on the mechanism by which such primer dimers form. One possible explanation is the extension of one primer over another through a tandem interaction within the catalytic site of DNA polymerases (FIG. 1).

Once formed, primer dimers amplify fast and may quickly deplete the pool of available reagents even before target amplification can be detected—especially if the target is present at a very low copy number.

As a result, primer dimer formation is one of the key factors limiting specificity, sensitivity, and yield in all nucleic acid amplification methods that use priming oligonucleotides, such as in Polymerase Chain Reaction (PCR), which is the most common method used. Apart from PCR, persistent primer dimer formation is particularly problematic in most, if not all, isothermal amplification methods. Examples include—without being limited to—Helicase-Dependent Amplification (HDA), Recombinase Polymerase Amplification (RPA), Loop-mediated Isothermal Amplification (LAMP), and Strand Displacement Amplification (SDA). The relatively high susceptibility of isothermal amplification reactions to being dominated by propagation of primer dimers can be in part explained by the fact that primer interactions are not continuously reset, as it happens through denaturation cycles in PCR. Another possible explanation is that in isothermal methods amplification speed is directly linked to DNA length, strongly favouring smaller products.

The method disclosed in US 2009/0258353 A1 uses a 5′ hybridisation cassette to encourage non-amplifiable primer dimerization however in this method the 5′ region is the reverse compliment of the start of the 3′ target specific region. This leads to either hairpin structures where the primer loops back on itself or dimers hybridised at the 5′ ends. The method of US 2009/0258353 A1 must, by design, be a sequence found in nature as it is the reverse compliment of a part of the target-specific region of the primer which is complementary to a natural sequence.

The method in WO 2015/164494 A1 uses, again, primers capable of dimerising through their 5′ regions. However, in this method the 5′ regions comprise a restriction site for a nicking enzyme. This site must therefore be a sequence found in nature.

In WO 2017/117287 A1 is disclosed the use of primers with 5′ extensions that are used to install new, highly specific primer binding sites into amplicons. These may be used for Tagged Amplicon Primer Extension (TAPE) or to increase confidence in a detection assay. While it is mentioned that the primers can form non-extendible dimers this is not the focus of the invention.

WO02016161054 A1 describes a method for fusing multiple nucleic acid sequences together using primers with complementary 5′ extensions. Multiple sets of primers are used to amplify target genes and a primer from each pair has a 5′ extension that is complementary to the extension of a primer from another pair. This creates amplicons that can be fused together into one strand. There is no non-replicable linker in this invention, as use of such would preclude their use for nucleic acid fusion as the 5′ extensions would not become incorporated.

The primers in WO 2017/165289 A1 feature a self-complementary 5′ extension such that they could form hairpin structures or non-extendible dimers. However these dimers are used for whole cell amplification due to the random or semi-random 3′ region. They are not targeted for a particular gene. In addition they do not contain a non-replicable linker region.

WO 94/21820 discloses a method of producing amplicons with free 5′ ends using primers with 5′ extensions and a non-replicable linker region. However the 5′ extensions are not specifically designed to hybridise and are used instead to detect the presence of the amplicon once it is produced.

### SUMMARY

One way of resolving the issue of primer dimer formation is to force the primers to dimerise in a mode that prevents them from being extended, such as by hybridisation of the 5′ ends. This kind of primer dimer cannot be extended as all commonly used nucleic acid polymerases have only 5′ to 3′ activity and the 3′ ends of the primer dimer have no template. The use of this technique can be improved by, for example the use of nucleic acid sequences not found in nature (nullomers) to provide the 5′ hybridisation cassettes and a linker region separating the 3′ target specific binding region of the primer from the 5′ hybridisation cassette which prevents incorporation of the 5′ hybridisation cassette into the amplicon.

Here, we propose a novel method to limit primer dimer formation during nucleic acid amplification by reversibly trapping the priming oligonucleotides in a molecular species called a Thermodynamic Trap (ThermoTrap). Thermodynamic Trap is very simple and cost-effective to implement. In contrast to other methods, ThermoTrap does not require modifying reaction chemistry—it works solely though using short artificial sequences added at the 5′ end of primers which hybridise to each other. The 5′ complementary regions of the primer are capable of hybridisation to other primers, but not the amplicon target sequences.

The method described includes a method for the amplification of nucleic acid sequences comprising:
a. taking a reaction mixture comprising:
   i. a nucleic acid sample;
   ii. a first nucleic acid amplification primer having a 3′ region which is complementary to a first target region of the sample and a 5′ region which is not complementary to a region of the sample; wherein the 5′ region is either self-complementary such that the 5′ ends of a first strand of the first nucleic acid amplification primer are capable of hybridising to the 5′ ends of a second strand of the first nucleic acid amplification primer, or the 5′ region of the first nucleic acid amplification primer is complementary to the 5′ region of a second nucleic acid amplification primer;
   iii. a nucleic acid polymerase;
   iv. nucleotide triphosphate monomers; and optionally
   v. a second nucleic acid amplification primer having a 3′ region which is complementary to an extension product of the first primer and a 5′ region which is complementary to the 5′ region of the first primer and is not complementary to a region of the sample;
b. hybridising the first primer to the sample,
c. extending the first primer using the nucleic acid polymerase and nucleotide triphosphate monomers; and
d. repeating steps b and c, thereby amplifying target sequences where the first nucleic acid amplification primer hybridises to the sample.

The method can be performed using a single species of the first amplification primer. In such cases the amplification is a linear amplification based on repeatedly hybridising and extending. The extended strands can be displaced using a strand displacing polymerase enzyme or similar enzymatic displacement.

Alternatively the amplification can include a second amplification primer, making the amplification exponential. The second primer copies the extension products from the first primers. The method of repeatedly hybridising and extending the first primers also repeatedly hybridises and extends the second primers, thus copying the copies.

The amplification can be isothermal, or can be carried out by thermocycling. Where the amplification is isothermal, the extended primer can be displaced from the sample using an enzyme, for example a helicase or recombinase. Where the amplification is performed by thermocycling, the extended primer is displaced from the sample using heat.

In order to provide a universal sequence that can be used on any sample, the 5′ region of the first nucleic acid amplification primer has a sequence which does not occur in nature. The 5′ region of the first nucleic acid amplification primer is either self-complementary or is complementary to the 5′ region of a second nucleic acid amplification primer. When the 5′ region of the first nucleic acid amplification primer has a sequence which does not occur in nature, then the complementary copy will also not occur in nature, hence the 5′ region of the second nucleic acid amplification primer will also have a sequence which does not occur in nature.

In order to function effectively, the 5′ non complementary region of the first nucleic acid amplification primer can have a lower melting temperature than the 3′ target complementary region of the first nucleic acid amplification primer.

In order to be universally applicable to any population of primers, the 5′ non complementary region of the first nucleic acid amplification primer can be palindromic. Where there are more than one primer species, the 5′ non complementary region of each of the amplification primers can be identical and palindromic Thus any member of the population can hybridise to any other member of the population of primers.

The 5′ and 3′ regions of the primer may be linked via a spacer unit which can not be copied by the polymerase. Suitable polymerase resistant spacer units include an alkyl (CH₂) chain or ethylene glycol (CH₂O) chain. The spacer unit could also be a modified nucleotide or ribonucleotide.

The method may be used in a multiplexed format with two or more first nucleic acid amplification primer of different target sequence.

Described also is a kit for the amplification of nucleic acid sequences comprising:
a. a first nucleic acid amplification primer having a 3′ region which is complementary to a first target region of the sample and a 5′ region which is not complementary to a region of the sample; wherein the 5′ region is either self-complementary such that the 5′ ends of a first strand of the first nucleic acid amplification primer are capable of hybridising to the 5′ ends of a second strand of the first nucleic acid amplification primer, or the 5′ region of the first nucleic acid amplification primer is complementary to the 5′ region of a second nucleic acid amplification primer;
b. a nucleic acid polymerase;
c. nucleotide triphosphate monomers; and optionally
d. a second nucleic acid amplification primer having a 3′ region which is complementary to an extension product of the first primer and a 5′ region which is complementary to the 5′ region of the first primer and is not complementary to a region of the sample.

The kit may comprise the second amplification primer.

The kit may comprise a helicase or recombinase.

The kit may comprise a first nucleic acid amplification primer wherein the 5′ region of the first nucleic acid amplification primer has a sequence which does not occur in nature.

The kit may comprise a first nucleic acid amplification primer wherein the 5′ non complementary region of the first nucleic acid amplification primer is palindromic.

The kit may comprise a first nucleic acid amplification primer wherein the 5′ non complementary region of the first nucleic acid amplification primer is attached to the primer via a spacer unit which can not be copied by the polymerase. Suitable polymerase resistant spacer units include an alkyl (CH₂) chain or ethylene glycol (CH₂O) chain. The spacer unit could also be a modified nucleotide or ribonucleotide.

The kit may contain more than one first nucleic acid amplification primer.

### FIGURES

FIG. 1. Primer interactions leading to formation and amplification of primer dimers. (A) Interaction with limited base pairing. (B) An example of an interaction without base pairing. Both types of interactions produce protruding 5′ DNA ends that serve as templates for strand extension from the 3′-end by DNA polymerases, all of which have a 3′-to-5′ directionality.

FIG. 2. The general principle of Thermodynamic Trap. (A) Interaction between complementary or partially complementary ThermoTrap elements located at primers 5′-ends (shown in diagonal stripes) forms a primer duplex with protruding 3′-ends, which cannot be extended by DNA polymerase. (B) In the amplification reaction mix, primer pairs are much more likely to be loaded into DNA polymerase catalytic site (gray circle) in a form of the ThermoTrap-mediated duplex rather than a 3′-to-3′ tandem with limited or no sequence complementarity. (C) In the presence of target sequences, these are bound by 3′-terminal primer regions with a higher affinity than the trapped thermolabile primer duplexes.

FIG. 3. Selected embodiments of the ThermoTrap design. (A) ThermoTrap elements are separated from the 3-terminal target-binding regions by an unrelated sequence. (B) ThermoTrap elements are positioned internally, flanked at the 5′ end by an unrelated sequence. (C) ThermoTrap elements are palindromic, allowing the primers to form both homo- and hetero-duplexes in any combination in a singleplex or multiplex reaction. (D) More than one ThermoTrap element may be attached to a primer to create higher-order primer complexes and further reduce the effective free molecule concentration in solution.

FIG. 4. Chemical separation of the ThermoTrap element and the target-binding region. (A) When the ThermoTrap element and the target-specific primer region are part of the same continuous nucleotide sequence, the ThermoTrap element becomes copied onto the complementary strand by extension of the template molecule from its 3′ end. (B) Introducing a spacer between the two sequences (ribbon) prevents propagation of the ThermoTrap sequence in subsequent rounds of amplification.

FIG. 5. Modification of 5′ primer end for enhanced performance in amplification reactions using DNA polymerases without strand-displacement activity. (A) Amplification with strand-displacing DNA polymerase leads to displacement of the trapped primer molecule through extension of the template molecule from its 3′ end. (B) When using DNA polymerase that lack strand displacement activity, such as Taq, trapped primer molecule can be degraded by the polymerase while extending the target 3′ end. (C) Chemically modifying the 5′ end o the ThermoTrap-containing primer (diamond symbol) prevents trapped primer degradation by DNA polymerases lacking strand displacement activity. For clarity, a ThermoTrap primer design is shown where the ThermoTrap element is separated from the 3′-terminal target-binding sequence by an unrelated sequence.

FIG. 6. Solution primer deposition with ThermoTrap primer design. (A) One of the primers form the primer pair is covalently or otherwise linked to a solid surface on its 5′ end. For clarity, a ThermoTrap primer design is shown with a ThermoTrap element positioned internally, flanked at the 5′ end by an unrelated sequence serving as a linker. (B) The solution primer is added and hybridized to the immobilized primer via the ThermoTrap element. (C) In an isothermal amplification reaction, following addition of amplification reagents the hybridized solution primer becomes displaced by DNA polymerase extending the 3′ end of bound template molecule.

FIG. 7. ThermoTrap sequences used as adapters in library generation. (A) Complementary ThermoTrap elements produce asymmetrically adapted libraries. (B) Palindromic ThermoTrap elements produce symmetrically adapted libraries.

FIG. 8. ThermoTrap primer design reduces the incidence of false-positive primer dimer amplification in Helicase-Dependent Amplification (HDA) assay. (A) Reaction amplification curves in absence of template molecules. Gel electrophoresis confirmed the identify of the products as primer dimers (not shown). (B) Reaction amplification curves in presence of template molecules.

FIG. 9. ThermoTrap primer design can prevent non-specific amplification of contaminant DNA. Amplification of E. coli uidA gene region in presence of either target DNA or defined amounts of human genomic DNA contaminant. Product amplification curves (left) and product melt curves (right) are shown. Solid lines depict PCR products amplified in presence of different input uidA target copy numbers (from 10 to 10.000 copies) in the absence of contaminating human genomic DNA (B01-F02 and B07-F08). Dotted lines depict PCR products amplified in presence of between 50 and 500 ng of contaminating human genomic DNA (G01, G02, G07, G08 and H01, H02, H07, H08) as well as no-template control (A01, A02 and A07, A08). (A) Amplification with standard primer design, where primers contain only target-binding sequence. (B) Amplification with Alpha8 ThermoTrap sequences conjugated with only target-binding sequence at their 5′ ends.

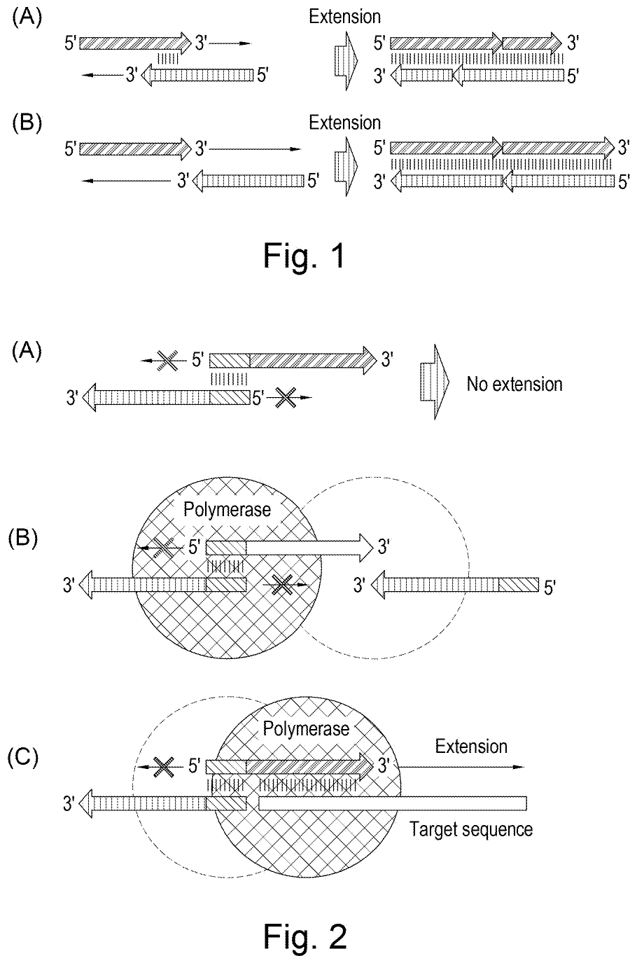

### DESCRIPTION

All known DNA polymerases have a strict enzymatic directionality, acting only at a hydroxyl group on the 3′ end of one DNA strand and adding nucleotides complementary to those present in a second DNA strand with a protruding 5′ end. Unwanted primer dimer amplification occurs through primer interactions forming a duplex with such protruding 5′ ends.

ThermoTrap primer design prevents undesired primer interactions by allowing the primers to reversibly interact with each other in an alternative way that does not result in formation of amplifiable dimers. This is achieved by adding to their 5′-ends relatively short and low-melting temperature DNA sequence(s) with a complete or partial complementarity (FIG. 2A). Interaction though these sequences, further referred to as ThermoTrap elements, leads to formation of a primer duplexes with protruding 3′ends, which cannot be amplified by DNA polymerases.

The 3′ end-part of the primer that is designed to interact with the target DNA sequence remains unaltered and fully exposed. Therefore, detrimental 3′-to-3′ primer dimer interaction could theoretically still occur at the same time as the ThermoTrap-mediated duplexes form (FIG. 2B). However, because DNA polymerase enzymes are relatively large compared to a DNA oligonucleotide, the two primer conformations compete for binding with the enzyme's catalytic site. Since the ThermoTrap elements are at least partially complementary, their loading is thermodynamically preferred, thus sterically preventing loading and extension of unwanted primer duplexes that share no or very limited complementarity.

Apart from steric interference, binding two primers together effectively reduces the molar concentration of primer molecules in solution by half, thereby thermodynamically reducing the probability of unwanted interactions. Since the target-specific part of the primer is longer and has higher melting temperature than the ThermoTrap element, binding target DNA sequences is thermodynamically more favourable than the ThermoTrap-mediated duplexes, therefore conferring the ability to amplify target sequences (FIG. 2C). Trapped primers are made available by spontaneously dissociating from the duplex during the amplification reaction.

Sequence Composition

Nucleotide sequences used as ThermoTrap elements can be selected from any naturally occurring sequences, but can also be partially or entirely artificial, to the extent that, in some embodiments, there might be no need to enzymatically copy these. Of benefit are artificial sequences not found in Nature (sequences showing no significant sequence similarity to any known sequence; also referred to as nullomers) and predicted or optimized to have minimal cross-reactivity with amplification of any natural target sequence.

A process to generate nullomers could be the following:
a. Generate a random population of primers satisfying pre-defined criteria. Criteria can be selected from any nucleotide sequence properties, such as length, percentage of guanines and cytidines (GC %), sequence melting temperature, Gibbs Free energy, tendency for formation of secondary structures or given dinucleotide composition.
b. From that pool, filter out sequences with a significant sequence similarity to naturally occurring sequences by applying an heuristic algorithm, such as—without being limited to—BLAST, BLAT or SSAHA2 on a non-redundant bank of all DNA sequences found in nature (such as NCBI NR databank).
c. Filter out sequences with a significant sequence similarity as shown by applying an EXACT algorithm, such as a Smith & Waterman local alignment method.
d. Filter out sequences with a significant binding affinity as shown by applying a thermodynamic simulation of annealing between primers and all positions of all sequences (e.g. based on Nearest-neighbour thermodynamic tables).

Sequences of ThermoTrap elements that are optimized for use with given target-specific binding regions can be identified with various bioinformatic as well as experimental methods. In the latter case, ThermoTrap sequences can be selected in a high throughput screen, where a random pool of ThermoTrap sequences attached to a common target-specific binding region is used in amplification under challenging condition, such as in presence of abundant contaminating DNA. On-target (specific) and off-target (non-specific) amplification products can be subsequently identified and quantified with next-generation sequencing (“NGS”), allowing selection of ThermoTrap sequences with the best on-target-to-off-target ratio.

Distance Between the ThermoTrap Region and the Target-Binding Region.

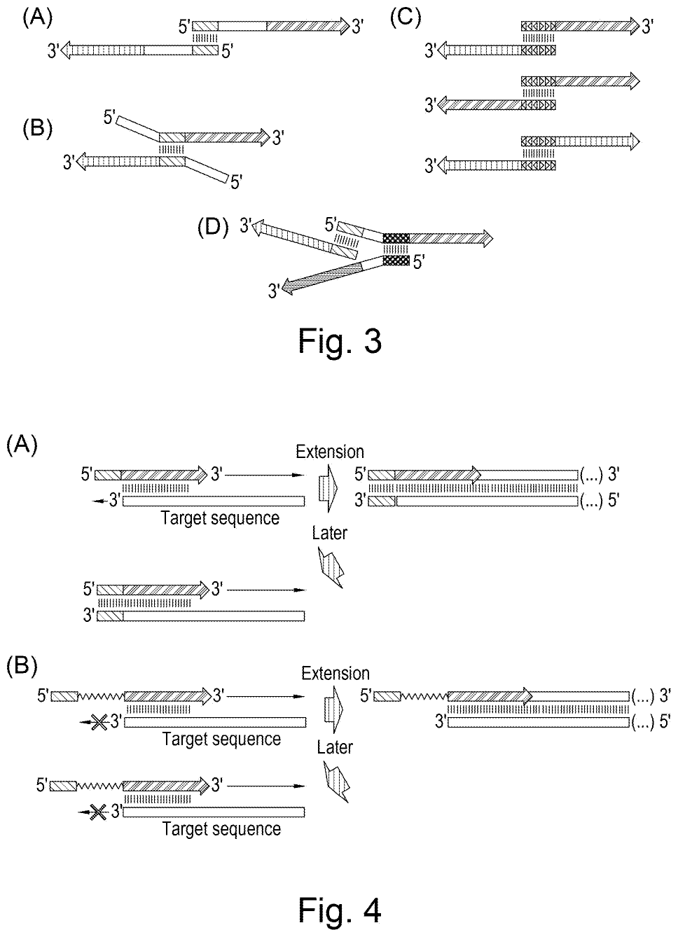

The mechanism of action outlined describes competition between ThermoTrap-mediated and non-specific primer duplexes for binding to DNA polymerase due to steric interference. Therefore, the distance between the ThermoTrap region and the 3′ primer end should be small enough to mediate such competition, i.e. prevent two DNA polymerase enzyme units to bind the two primer ends independently (FIG. 2 depicts the two elements as immediately adjacent). However, even if this distance is too large to allow for competition, presence of such separated ThermoTrap regions still brings a benefit by reducing the molar concentration of free primer molecules in solution. Therefore, included within this disclosure are primer designs regardless of the distance between the ThermoTrap region and the 3′-terminal target-specific region, as long as they are located on the same oligonucleotide molecule (FIG. 3A).

Position of the ThermoTrap Regions

FIG. 2A depicts the ThermoTrap element positioned directly at the 5′ primer end. However, ThermoTrap element may also be located internally, with any DNA sequence proceeding it at the 5′ end (FIG. 3B). For example, in applications such as Helicase-Dependent Amplification, ThermoTrap elements may be flanked at the 5′ end by a low melting-temperature sequence to promote DNA unwinding by the helicase enzyme.

Complementarity of the ThermoTrap Regions

FIG. 2A depicts a design in which ThermoTrap sequences are fully complementary. However, the ThermoTrap elements may also be designed to include mismatches or modified bases.

Furthermore, FIG. 2A depicts a design in which ThermoTrap sequences are attached to two different primers such that primer hetero-duplexes are formed. However, ThermoTrap sequences may also be designed as palindromic (sequences on both complementary strands which are identical when read in 5′-to-3′ direction). Palindromic ThermoTrap elements can form both homo- and hetero-duplexes in all combinations with any other ThermoTrap-containing primers present in the reaction (FIG. 3C). Therefore, palindromic design allows employment of a single ThermoTrap element design in complex primer pools of multiplex reactions.

ThermoTrap tail length can be adjusted to optimize the balance between amplification efficiency and primer dimer inhibition. Long ThermoTrap tails are predicted to confer high resistance to primer dimer formation at the cost of lower amplification efficiency, while short ThermoTrap tails are expected to confer higher amplification efficiency at the cost of lower resistance to primer dimer formation.

Higher-Order Combinations

FIG. 2A depicts a design in which each primer contains one ThermoTrap element. However, in fact more than one different or the same ThermoTrap elements may be attached to a single primer to facilitate formation of higher-order primer complexes (i.e. with more than two primers). Such design would allow to further reduce the concentration of free primer molecules in the reaction (FIG. 3D).

Chemical Separation

When the ThermoTrap element and the target-specific primer region are part of the same continuous nucleotide sequence, the ThermoTrap sequence becomes incorporated onto the products complementary strand by extension of the template molecule from its 3′ end (FIG. 4A). Subsequently, the presence of the ThermoTrap sequence will influence thermodynamic properties of the primer binding to its target in the subsequent rounds of amplification, such as increasing their annealing temperature or increasing probability of mispriming. To overcome this, the ThermoTrap element may be chemically separated during oligonucleotide synthesis from the target-specific primer region by one or several linker or blocking molecules, such as—without being limited to—hexanediol, ethyleneglycols, pyrene or phosphoramidites (FIG. 4B). RNA or modified nucleotides, such as locked nucleic acids (LNA) or peptide nucleic acids (PNA) can also be used. In this embodiment, chemical separation prevents propagation of the ThermoTrap sequence in the amplified target DNA, while retaining the physical attachment of the two primer elements.

In addition to retaining primers melting temperature and reducing mispriming, chemically separating ThermoTrap element and the target-specific primer region also reduces consumption of amplification reagents, such as deoxyribonucleotide triphosphates (dNTPs), as well as allows for more flexibility in the design of longer or more complex ThermoTrap region without interfering with target amplification.

Modified 5′ Ends for Enhanced Performance in Amplification Reactions Using DNA Polymerases Without Strand-Displacement Activity

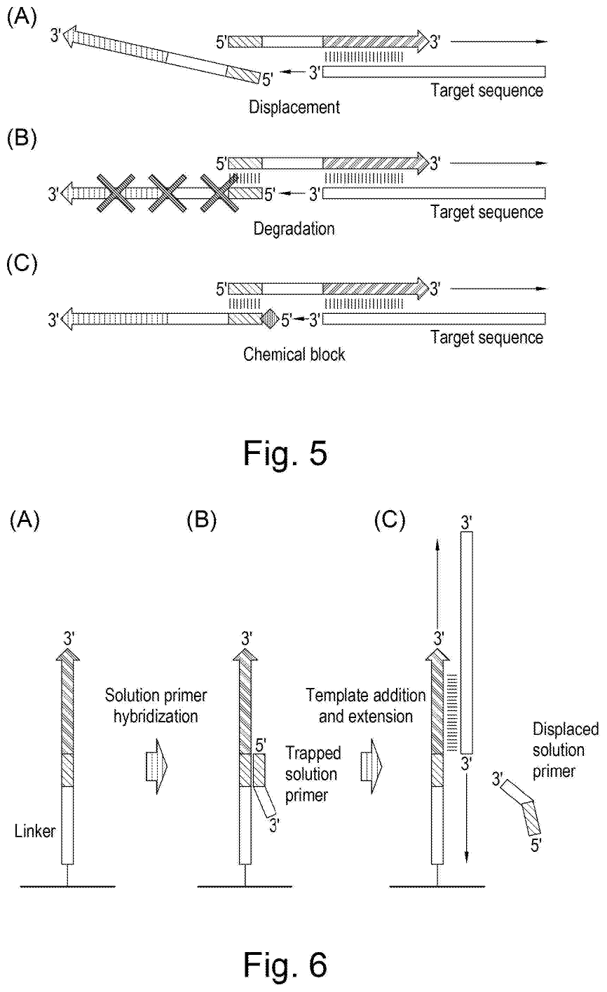

Most isothermal amplification methods use DNA polymerases with a strand displacement activity, which allows for the trapped primer to be dislocated during the extension step (FIG. 5A). If ThermoTrap design is used in amplification methods that utilize polymerases with 5′ exonuclease activity, such as Taq polymerase used in PCR, the trapped primers could be degraded by the extending polymerase (FIG. 5B).

In order to avoid this, 5′ end of ThermoTrap-containing primers can be modified with a moiety that protects it from 5′-directed exonuclease activity, such as—but not limited to—a phosphorothioate bond, non-nucleotides or modified nucleotides. Modification would block the extension of the template molecule over the ThermoTrap element, thus also providing the benefits of a chemically separated ThermoTrap and priming regions.

Primer Deposition in Solid-Phase Amplification

When applied to assays based on solid-phase amplification, where one of the primers from the primer pair is immobilized to a solid surface by its 5′ end, ThermoTrap primer design can be used to deposit the solution primer molecules in situ prior to addition of the template and initiation of the reaction (FIG. 6). This can be achieved by pre-hybridizing the solution primer to the surface decorated with the immobilized primer, such as on the bottom of a micro-well, before reaction mix is added. Subsequently, binding and extension of the template DNA would release the trapped solution primer and allow it to bind at the other, distal site of the template molecule. In isothermal amplification, this allows to (1) concentrate solution primer close to where it is needed, (2) achieve a higher total amount of primer available in the reaction, (3) gradually release the primer during the reaction, as it becomes needed.

ThermoTrap Elements Used as Adapters in Library Generation

Many highly multiplexed molecular biology assays that involve nucleic acid amplification, such as—but not limited to—targeted next-generation sequencing (NGS) or assays of amplification products (known as amplicon sequencing), require generation of libraries of amplified template molecules. Such libraries can be generated through a massively multiplex PCR reaction containing hundreds of primer pairs. One important step in library preparation for NGS applications is addition of universal adapters that allow to uniformly amplify the library with a single pair of PCR primers in a subsequent PCR reaction. Adapters may also contain a priming site for the sequencing primer and sequences that facilitate binding of library molecules to the surface of a sequencing chip. In addition, adapters may also contain so called indices that serve as barcodes enabling the user to mix different samples within one sequencing reaction and later deconvolute their origin.

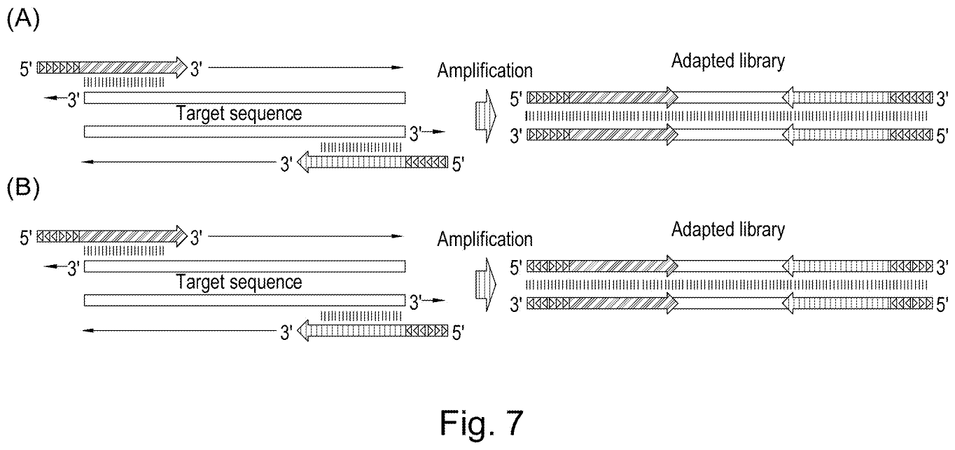

When used in such highly multiplex assays, ThermoTrap sequences may be employed to serve a dual function of ThermoTrap elements and universal adaptors (as depicted on FIG. 3C). Using two complementary ThermoTrap sequences, one for each of the two primers in all primer pairs, will result in generation of a universally adapted library with two complementary adapters on both ends (FIG. 7A). Using a single identical palindromic ThermoTrap sequence conjugated with all primers used in a multiplex reaction will result in generation of a universally and symmetrically adapted library (FIG. 7B).

### EXPERIMENTS

### Experiment 1

ThermoTrap Primer Design in Helicase-Dependent Amplification (HDA)

Methods:

Efficiency of ThermoTrap primer design in reducing the incidence of primer dimers during an isothermal amplification was tested in a singleplex Helicase-Dependent Amplification (HDA) assay using IsoAmp III Universal tHDA® chemistry (Biohelix Corp). All primers were designed such that they contained common target-specific binding regions AAAACGAGACATGCCGAGCATCCGC and AAAAACTCCTCTGGCACCGTGCTGC at their 3′ ends, labelled as HDA72_F and HDA72_R for the forward and reverse primers, respectively. At their 5′ ends primers contained either no additional sequence (control primers) or one of the four variants a non-palindromic ThermoTrap element:
(1) Alpha8 variant ACTGACGT (or its complementary sequence ACGTCAGT in the HDA72_R reverse primer),
(2) A Alpha8 variant AAAAACTGACGT, which contained four additional adenines at the 5′ end (or its complementary sequence with four additional adenines, AAAAACGTCAGT, in the HDA72_R reverse primer),
(3) T_Alpha8 variant TTTTACTGACGT, which contained four additional thymidines at the 5′ end (or its complementary sequence with four additional thymidines, TTTTACGTCAGT, in the HDA72_R reverse primer),
(4) Alpha16 variant ACTGACGTGATCTGCA, were a 16 nucleotide-long non-palindromic ThermoTrap element was used instead of the 8 nucleotide-long Alpha8 element (or its complementary sequence TGCAGATCACGTCAGT in the HDA72_R reverse primer).

See Table 1 for details.

25 μl reactions were prepared containing 1× Annealing Buffer II, 0.3×SYBR Green, 1 μl Enzyme Mix, 1.75 μl dNTP Mix, 4 mM MgSO₄, 40 NaCl and 75 nM of a forward and reverse primer for each of the five primer pairs. 10\hat{ }8 copies of template molecules containing HDA72_F and HDA72_R sequences at their ends (template-containing reactions) or water (NTCs, no-template controls) were added to each reaction. 16 replicate reactions were prepared for each of the 5 primer pairs, 8 with template and 8 NTCs. Reactions were incubated at 65° C. for 2 hours in QuantStudio 7 Flex Real-Time PCR System (Thermo Fisher Scientific). Increase in product fluorescence was monitored in 1-minute intervals. Fluorescence data was plotted as a function of time.

Results:

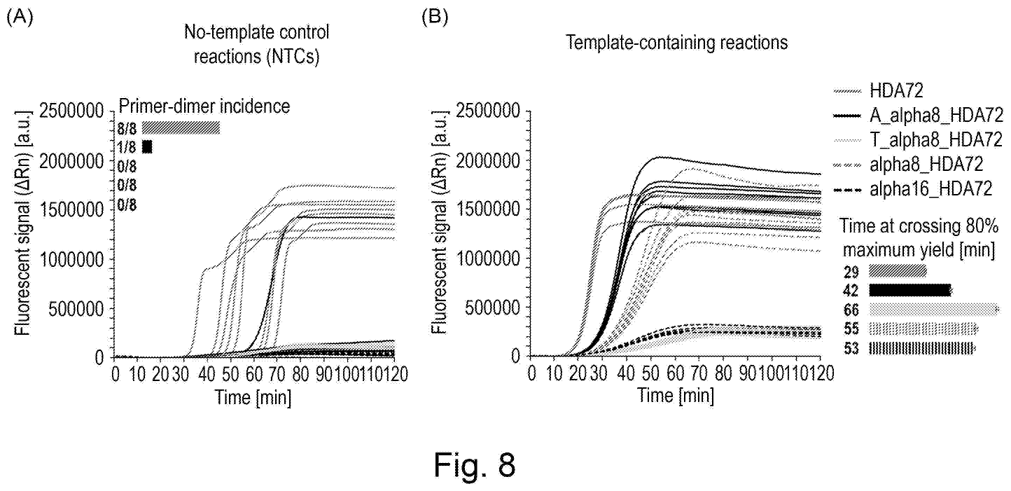

ThermoTrap primer design prevented primer dimer amplification in the absence of template DNA. ThermoTrap design A_Alpha8, with an 8 nucleotide-long trap sequence and 4 free adenines at the 5′ end reduced dimer incidence by 87.5% but simultaneously increased amplification time by 45%. Alpha8 design without any flanking sequence at the 5′ end reduced the primer dimer incidence to 0 but increased amplification time by 127%. Other designs significantly impaired target amplification yield (FIG. 8).

### Experiment 2

ThermoTrap Primer Design in Polymerase Chain Reaction (PCR)

Efficiency of ThermoTrap primer design in reducing non-specific amplification in Polymerase Chain Reaction (PCR) was tested in a singleplex assay designed to amplify a region of E. coli uidA gene either in presence of target DNA or varying amounts of human genomic DNA contaminant.

Two primer pairs were compared that contained target-specific binding sequences at their 3′ ends (Ecol_uidA_1_379_20_Par: AGTTGCAACCACCTGYTGAT, Ecol_uidA_1_80_22_PAf: GTATGTTATTGCCGGGAAAAGT) but differed in presence of absence of an 8 nucleotide-long Alpha8 ThermoTrap sequence at their 5′ ends (ACTGACGT and ACGTCAGT in the forward and reverse primer, respectively).

20 μl PCR reactions were prepared containing 0.125× Titanium PCR Buffer (ClonTech), 2.67 mM MgCl₂, 48 mM KCl, 0.32×SYBR Green, 0.2 mM dNTPs each, 1× Titanium Taq Polymerase (Clontech) and 0.2 μM each primer from a primer pair.

Reactions were prepared containing either no template, 50 or 500 ng of human genomic DNA extract, or between 10 to 10.000 copies of uidA target copies per reaction. Reactions were set up in duplicates with either of the two primer pairs tested. PCR has been performed in a thermocycler with a real-time fluorescence reading for 40 amplification cycles using following program:

| Temperature | Time (s) | Cycles |
|---|---|---|
| 95° C. | 120 | — |
| 95° C. | 10 | 40 |
| 62° C. | 10 | |
| 72° C. | 30 | |

Increase in product fluorescence was monitored and fluorescence data was plotted as a function of time. After amplification products were subjected to melt curve analysis to differentiate between on-target (approx. 89° C. melting temperature) and off-target (melting temperature below 89° C.) amplification products.

Results:

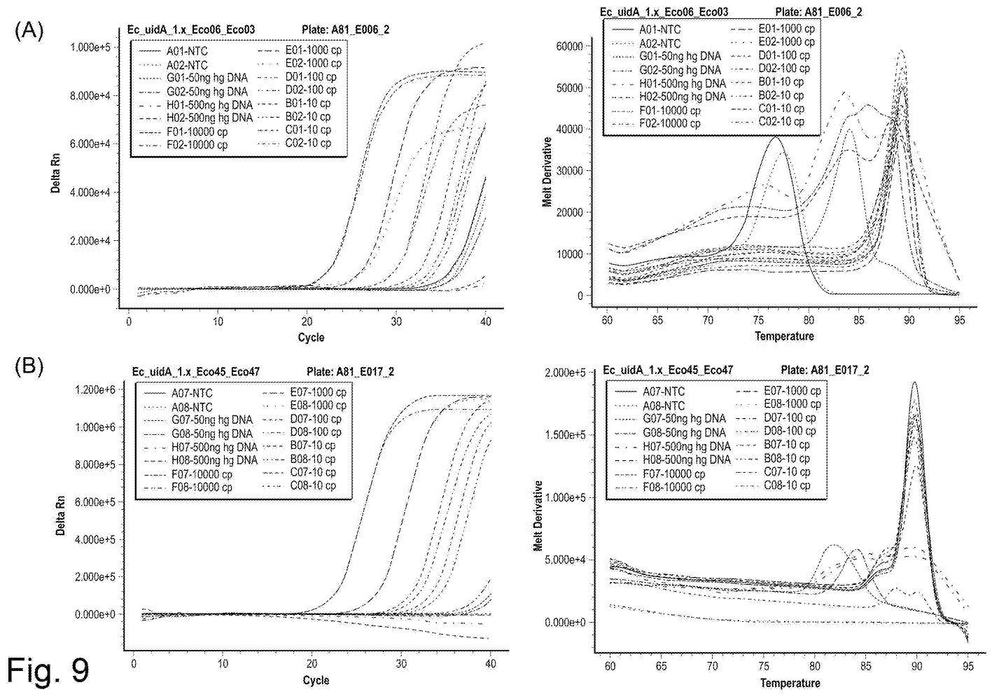

Primers lacking ThermoTrap sequence showed significant off-target amplification in presence of human genomic contaminant, while primers containing the Alpha8 ThermoTrap design showed delayed or absent off-target amplification. At the same time, presence of the Alpha8 ThermoTrap sequences did not affect the sensitivity and speed of target amplification (FIG. 9).

1. A method for the amplification of nucleic acid sequences comprising:
a. taking a reaction mixture comprising:
i. a nucleic acid sample;
ii. a first nucleic acid amplification primer having a 3′ region which is complementary to a first target region of the sample and a 5′ region which is not complementary to a region of the sample and has a sequence which does not occur in nature; wherein the 5′ region is either self-complementary such that the 5′ ends of a first strand of the first nucleic acid amplification primer are capable of hybridising to the 5′ ends of a second strand of the first nucleic acid amplification primer, or the 5′ region of the first nucleic acid amplification primer is complementary to the 5′ region of a second nucleic acid amplification primer, and wherein the 5′ non complementary region of the first nucleic acid amplification primer is attached to the primer via a spacer unit which can not be copied by the polymerase;
iii. a nucleic acid polymerase;
iv. nucleotide triphosphate monomers; and optionally
v. a second nucleic acid amplification primer having a 3′ region which is complementary to an extension product of the first primer and a 5′ region which is complementary to the 5′ region of the first primer and is not complementary to a region of the sample;
b. hybridising the first primer to the sample,
c. extending the first primer using the nucleic acid polymerase and nucleotide triphosphate monomers; and
d. repeating steps b and c, thereby amplifying target sequences where the first nucleic acid amplification primer hybridises to the sample.

2. The method according to claim 1 wherein the amplification is carried out with only a first amplification primer.

3. The method according to claim 1 wherein the amplification is carried out with the second amplification primer.

4. The method according to any one of claims 1-3 wherein the amplification is isothermal.

5. The method according to claim 4 wherein the extended primer is displaced from the sample using an enzyme.

6. The method according to claim 5 wherein the enzyme is a helicase or recombinase.

7. The method according to any one of claims 1-3 wherein the amplification is thermocycling.

8. The method according to any one preceding claim wherein the 5′ non complementary region of the first nucleic acid amplification primer has a lower melting temperature than the 3′ target complementary region of the first nucleic acid amplification primer.

9. The method according to any one preceding claim wherein the 5′ non complementary region of the first nucleic acid amplification primer is palindromic.

10. The method according to any one preceding claim wherein the 5′ non complementary region of the first and second nucleic acid amplification primers are identical and are palindromic.

11. The method according to claim 1 wherein the spacer unit is an alkyl (CH₂) chain or ethylene glycol (CH₂O) chain.

12. The method according to claim 1 wherein the spacer unit is a modified nucleotide or ribonucleotide.

13. The method according to any one preceding claim wherein the method uses more than one first nucleic acid amplification primer.

14. The method according to any one preceding claim wherein the method uses more than one second nucleic acid amplification primer.

15. A kit for the amplification of nucleic acid sequences comprising:
a. a first nucleic acid amplification primer having a 3′ region which is complementary to a first target region of the sample and a 5′ region which is not complementary to a region of the sample and has a sequence which does not occur in nature; wherein the 5′ region is either self-complementary such that the 5′ ends of a first strand of the first nucleic acid amplification primer are capable of hybridising to the 5′ ends of a second strand of the first nucleic acid amplification primer, or the 5′ region of the first nucleic acid amplification primer is complementary to the 5′ region of a second nucleic acid amplification primer, and wherein the 5′ non complementary region of the first nucleic acid amplification primer is attached to the primer via a spacer unit which can not be copied by the polymerase;
b. a nucleic acid polymerase;
c. nucleotide triphosphate monomers; and optionally
d. a second nucleic acid amplification primer having a 3′ region which is complementary to an extension product of the first primer and a 5′ region which is complementary to the 5′ region of the first primer and has a sequence which does not occur in nature and is not complementary to a region of the sample.

16. The kit according to claim 15 further comprising the second amplification primer.

17. The kit according to claim 15 further comprising a helicase or recombinase.

18. The kit according to any one of claims 15-17 wherein the 5′ region of the first nucleic acid amplification primer has a sequence which does not occur in nature.

19. The kit according to any one of claims 15-18 wherein the 5′ non complementary region of the first nucleic acid amplification primer is palindromic.

20. The kit according to any one of claims 15-19 wherein the spacer unit is an alkyl (CH₂) chain or ethylene glycol (CH₂O) chain.

21. The kit according to any one of claims 15-19 wherein the spacer unit is a modified nucleotide or ribonucleotide.

22. The kit according to any one of claims 15-21 wherein the kit contains more than one first nucleic acid amplification primer.

23. The kit according to claim 22 wherein the kit contains more than one second nucleic acid amplification primer, wherein the 5′ non complementary region of the first and second nucleic acid amplification primers are identical and are palindromic.


SEQUENCE LISTING (22 sequences)

SEQ ID NO: 1 - DNA, 25 bp, Artificial Sequence
  HDA72_F
aaaacgagacatgccgagcatccgc

SEQ ID NO: 2 - DNA, 25 bp, Artificial Sequence
  HDA72_R
aaaaactcctctggcaccgtgctgc

SEQ ID NO: 3 - DNA, 8 bp, Artificial Sequence
  Alpha8 variant for forward HDA72_F primer
actgacgt

SEQ ID NO: 4 - DNA, 8 bp, Artificial Sequence
  Alpha8 variant for reverse HDA72_R primer
acgtcagt

SEQ ID NO: 5 - DNA, 12 bp, Artificial Sequence
  A_Alpha8 variant for forward HDA72_F primer
aaaaactgacgt

SEQ ID NO: 6 - DNA, 12 bp, Artificial Sequence
  A_Alpha8 variant for reverse HDA72_R primer
aaaaacgtcagt

SEQ ID NO: 7 - DNA, 12 bp, Artificial Sequence
  T_Alpha8 variant  for forward HDA72_F primer
ttttactgacgt

SEQ ID NO: 8 - DNA, 12 bp, Artificial Sequence
  T_Alpha8 variant  for reverse HDA72_R primer
ttttacgtcagt

SEQ ID NO: 9 - DNA, 16 bp, Artificial Sequence
  Alpha16 variant for forward HDA72_F primer
actgacgtgatctgca

SEQ ID NO: 10 - DNA, 16 bp, Artificial Sequence
  Alpha16 variant for reverse HDA72_R primer
tgcagatcacgtcagt

SEQ ID NO: 11 - DNA, 33 bp, Artificial Sequence
  Alpha8  forward HDA72_F primer
actgacgtaaaacgagacatgccgagcatccgc

SEQ ID NO: 12 - DNA, 33 bp, Artificial Sequence
  Alpha8  reverse HDA72_R primer
acgtcagtaaaaactcctctggcaccgtgctgc

SEQ ID NO: 13 - DNA, 37 bp, Artificial Sequence
  A_Alpha8  forward HDA72_F primer
aaaaactgacgtaaaacgagacatgccgagcatccgc

SEQ ID NO: 14 - DNA, 37 bp, Artificial Sequence
  A_Alpha8 reverse HDA72_R primer
aaaaacgtcagtaaaaactcctctggcaccgtgctgc

SEQ ID NO: 15 - DNA, 37 bp, Artificial Sequence
  T_Alpha8  forward HDA72_F primer
ttttactgacgtaaaacgagacatgccgagcatccgc

SEQ ID NO: 16 - DNA, 37 bp, Artificial Sequence
  T_Alpha8 reverse HDA72_R primer
ttttacgtcagtaaaaactcctctggcaccgtgctgc

SEQ ID NO: 17 - DNA, 41 bp, Artificial Sequence
  Alpha16  forward HDA72_F primer
actgacgtgatctgcaaaaacgagacatgccgagcatccgc

SEQ ID NO: 18 - DNA, 41 bp, Artificial Sequence
  Alpha16 reverse HDA72_R primer
tgcagatcacgtcagtaaaaactcctctggcaccgtgctgc

SEQ ID NO: 19 - DNA, 20 bp, Artificial Sequence
  Ecol_uidA_1_379_20_Par
agttgcaaccacctgytgat

SEQ ID NO: 20 - DNA, 22 bp, Artificial Sequence
  Ecol_uidA_1_80_22_Paf
gtatgttattgccgggaaaagt

SEQ ID NO: 21 - DNA, 28 bp, Artificial Sequence
  Alpha8 reverse Ecol_uidA_1_379_20_Par
acgtcagtagttgcaaccacctgytgat

SEQ ID NO: 22 - DNA, 30 bp, Artificial Sequence
  Alpha8 forward Ecol_uidA_1_80_22_Paf
actgacgtgtatgttattgccgggaaaagt